(19) United States
(12) Patent Application Publication
(10) Pub. No.: US 2009/0121155 A1
(43) Pub. Date: May 14, 2009

(54) RADIOTHERAPY APPARATUS AND PARTS THEREOF

(75) Inventors: Kevin Brown, Horsham (GB); Paul Boxall, Maidenbower (GB); Christopher James Gibson, Reigate (GB)

Correspondence Address: LAHIVE & COCKFIELD, LLP;FLOOR 30, SUITE 3000, ONE POST OFFICE SQUARE, BOSTON, MA, 02109, US

(73) Assignee: Elekta AB (publ), Stockholm (SE)

(21) Appl. No.: 11/937,764

(22) Filed: Nov. 9, 2007

(51) Int. Cl. A61N 5/00

(52) U.S. Cl. 250/492.1

(57) ABSTRACT

A geometry item (such as a gantry arm or an MLC leaf) of a radiotherapeutic apparatus needs to be moved in an accurate manner. The effect of inertia introduces a potential inaccuracy. A radiotherapeutic apparatus is therefore disclosed, comprising a geometry item, a radiation source capable of emitting a beam of therapeutic radiation, and a control unit, the geometry item being moveable to adjust the geometry of the beam, the radiation source having a variable dose rate, and the control unit being arranged to cause variations in the speed of movement of the geometry item and to adjust the dose rate of the radiation source for a period of time after a change in the speed of the geometry item.

### FIELD OF THE INVENTION

The present invention relates to improvements in or relating to radiotherapeutic apparatus.

### BACKGROUND ART

Radiotherapy is a process whereby a beam of harmful radiation is directed generally towards a region of a patient, usually in order to treat a tumour within that region. The radiation causes damage to living cells in its path, and hence inhibits or reduces the tumour. It also damages healthy tissue if applied in significant doses, and therefore efforts are made to limit the dose to healthy tissues while maintaining the prescribed dose to cancerous tissue.

One apparently straightforward means of limiting the dose to healthy tissue is to direct the beam towards the tumour from a plurality of different directions. Thus, the total dose delivered to the tumour can be significantly greater than the dose applied to the surrounding tissue. A common approach to doing so is to mount the radiation source on an arm (or gantry) extending from a rotateable support, with the arm located at a position offset from the axis of rotation and the source being oriented towards that axis so that the beam intersects with the axis. Thus, as the support rotates, the beam always passes through the point of intersection (usually referred to as the “isocentre”) but does so from every radial direction around the isocentre. This requires the gantry and the source to be rotated around the patient; both items have a significant mass and therefore the engineering challenge that this presents is significant.

Another means of limiting the dose applied to healthy tissue is the so-called “multi-leaf collimator” or “MLC” as shown in, for example, EP-A-314,214. An plurality of long narrow leaves are arranged side-by side in an array, and are individually controllable via a servo-motor so that they can each be extended or retracted by a desired amount. Thus, by moving individual leaves, a collimator can be made to a desired shape. A pair of such collimators, one either side of the beam, allows the beam to be shaped as desired thereby allowing healthy tissue to be placed in shadow.

In a multi-leaf collimator, the leaves are generally thin in the direction transverse to the direction of movement, to provide a good resolution, and long in the direction of movement so as to provide a good range of movement. In the direction of the beam, the leaves need to be relatively deep; even when made of a high atomic number material such as Tungsten, such depth is required in order to offer an adequate attenuation of the beam. Thus, leaves are relatively heavy and difficult to move.

### SUMMARY OF THE INVENTION

Both of these aspects of a radiotherapy apparatus require the relevant geometry item (in this case the gantry arm and the MLC leaves) to be moved during treatment in an accurate manner. Older “stop and shoot” methods called for the geometry item to be moved to a specific location, which can be checked easily by known servo-control methods. However, to improve treatment times, more modern treatment control methods call for the geometry item to be moved at a specific (linear or rotational) speed over a specific time period, after which it is moved at a (potentially) different speed for a further time period. This raises the issue of inertia.

Specifically, if a treatment plan calls for the geometry item to move at a particular speed v₁ over a time period t₁ followed by a speed v₂ over a subsequent time period t₂, it is not accurate to assume that the item will change its speed immediately. Instead, there will be a catch-up period during which the actual speed will be incorrect, either too high if v₁>v₂ or too low if v₁<v₂. In either case, the geometry item will be at an incorrect location during delivery of at least part of the dose.

The present invention therefore provides a radiotherapeutic apparatus comprising a geometry item, a radiation source capable of emitting a beam of therapeutic radiation, and a control unit, the geometry item being moveable to adjust the geometry of the beam, the radiation source having a variable dose rate, and the control unit being arranged to cause variations in the speed of movement of the geometry item and to adjust the dose rate of the radiation source for a period of time after a change in the speed of the geometry item.

The invention is applicable to various geometry items in a typical radiotherapeutic apparatus. Gantry arms often have a significant inertia, in that they carry a radiation source, itself having a significant inertial mass, and therefore have the required physical rigidity and the inertial mass that results therefrom. In their rotation about an axis offset from the arm, they will therefore exhibit a detectable inertia. Another example is the leaves of a multi-leaf collimator, which are relatively heavy in themselves, but also suffer from limitations as to the power with which they can be accelerated as a result of space limitations on the associated drive means and limits imposed by the need to prevent buckling of the leaves.

Radiation sources in the form of linear accelerators usually emit a pulsed beam of radiation, whose dose rate can be varied by varying the repetition frequency of the pulses in the pulsed beam.

### BRIEF DESCRIPTION OF THE DRAWINGS

An embodiment of the present invention will now be described by way of example, with reference to the accompanying figures in which;

FIG. 1 shows a typical variation of speed with time for a geometry item; and

FIG. 2 shows the resulting variation of distance or displacement with time for the item, compared with a ideal inertia-less item.

### DETAILED DESCRIPTION OF THE EMBODIMENTS

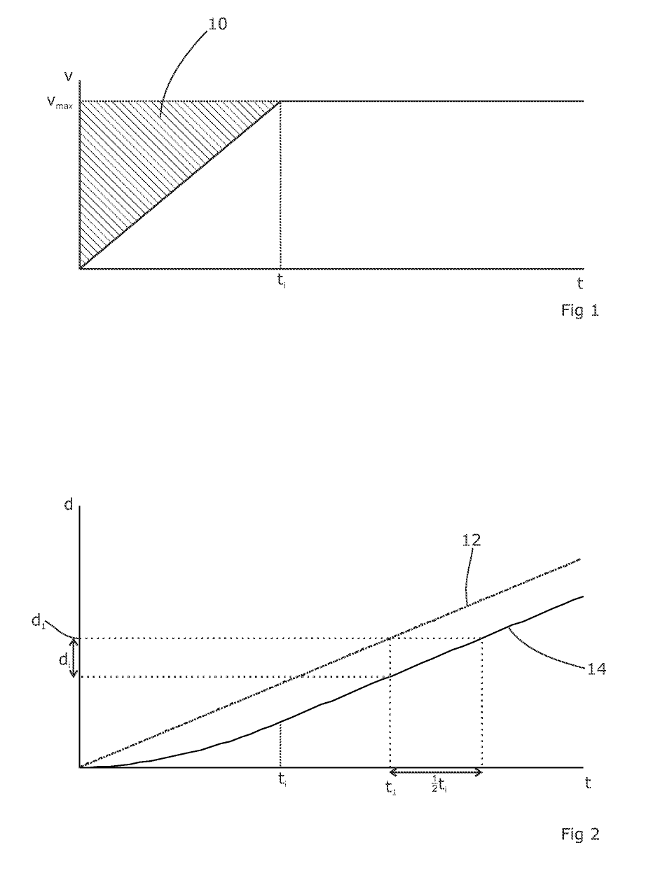

Referring to the accompanying figures, FIG. 1 shows an achievable velocity/time profile for a geometry item for a geometry item of a radiotherapy apparatus that suffers from a detectable inertia. Whilst it would in theory be assumed that the intended velocity vₘₐₓ would be achieved immediately, in practice it takes a certain amount of time tᵢ to overcome the inertia of the geometry item and raise its speed from rest (or its initial speed, if different) to vₘₐₓ. In the period prior to time tᵢ, therefore, the speed v of the item will increase lineally assuming that the maximum possible acceleration is being applied until it reaches vₘₐₓ at which point the control system will stop accelerating the item and continue to run it at its intended speed vₘₐₓ. This does, however, leave the geometry item lagging behind the idealised position by a distance which corresponds to the area 10.

FIG. 2 shows the corresponding distance-time graph. The path of an idealised item is shown as the straight line 12 increasing steadily at constant gradient corresponding to vₘₐₓ. The path of an item suffering from inertia is shown as line 14 and this initially lags behind line 12 as the speed of the item grows. Eventually, at time tᵢ, the maximum speed vₘₐₓ is reached and the item continues at a steady gradient corresponding to vₘₐₓ. At this point, however, it is behind the idealised line 12 by an amount which then remains substantially constant.

Thus, when the idealised geometry item has reached a distance d₁, the actual item 14 lags behind by a distance dᵢ. Alternatively, the actual item 14 arrives at the point d₁ after a delay of ½tᵢ or thereabouts, subject to the accuracy of the driving mechanism.

As a result, inertia compensation for the geometry item concerned can actually be achieved straightforwardly. Given that after time tᵢ, the item is moving in exactly the same way that a simple linear relationship would predict, the intended dose can thereafter be delivered in exactly the same way, albeit delayed by a time tᵢ/2. In the period up to tᵢ, the dose rate can be reduced to reflect the slower than expected movement of the geometry item. That reduction can be graded to the current speed or position of the item, or it can be based upon a suitably close approximation such as a halving of the dose rate to reflect the fact that, on average, the speed of the geometry item during this period is half way between its initial speed (0) and its final speed (Vₘₐₓ). Alternatively, more complex relationships between the dose rate and time tᵢ can be provided for depending on the accuracy that is required. For example, the dose rate could step up in two or more increments during this period, or could be slaved to a detected position or speed of a geometry item, or could follow a time-dependent uptake pattern calculated to replicate the acceleration of the geometry item concerned.

It will of course be understood that many variations may be made to the above-described embodiment without departing from the scope of the present invention.

1. Radiotherapeutic apparatus comprising a geometry item, a radiation source capable of emitting a beam of therapeutic radiation, and a control unit, the geometry item being moveable to adjust the geometry of the beam, the radiation source having a variable dose rate, and the control unit being arranged to cause variations in the speed of movement of the geometry item and to adjust the dose rate of the radiation source for a period of time after a change in the speed of the geometry item.

2. Radiotherapeutic apparatus according to claim 1 in which the geometry item is a gantry arm, the arm carrying a radiation source and being rotateable about an axis offset from the arm.

3. Radiotherapeutic apparatus according to claim 1 in which the geometry item is at least one leaf of a multi-leaf collimator.

4. Radiotherapeutic apparatus according to claim 1 in which the radiation source emits a pulsed beam of radiation.

5. Radiotherapeutic apparatus according to claim 1 in which the dose rate of the radiation source is variable by varying the repetition frequency of the pulses in the pulsed beam.
